(19) United States
(12) Patent Application Publication
(10) Pub. No.: US 2010/0112653 A1
(43) Pub. Date: May 6, 2010

(54) Process of Producing a Fermentation Product

(75) Inventors: Jiyin Liu, Raleigh, NC (US); Chee-Leong Soong, Raleigh, NC (US)

Correspondence Address: NOVOZYMES NORTH AMERICA, INC., 500 FIFTH AVENUE, SUITE 1600, NEW YORK, NY, 10110, US

(73) Assignee: NOVOZYMES NORTH AMERICA, INC., Franklinton, NC (US)

(21) Appl. No.: 12/598,321

(22) PCT Filed: May 9, 2008
(86) PCT No.: PCT/US08/63179

§ 371 Date: Oct. 30, 2009

Related U.S. Application Data
(60) Provisional application No. 60/916,988, filed on May 9, 2007.
(60) Provisional application No. 60/941,424, filed on Jun. 1, 2007.

(51) Int. Cl. C12P 7/04

(52) U.S. Cl. 435/157

(57) ABSTRACT

The present invention relates to processes for producing a fermentation product from starch-containing material comprising: (a) saccharifying starch-containing material with an alpha-amylase at a temperature below the initial gelatinization temperature of said starch-containing material; (b) fermenting using a fermenting organism, wherein a phytase is present during steps (a) and/or (b).

### BACKGROUND OF THE INVENTION

1. Field of the Invention

The present invention relates to processes of using a phytase in a process for producing a fermentation product, such as ethanol from un-gelatinized starch-containing material (i.e., uncooked starch-containing material).

2. Description of the Related Art

A vast number of commercial fermentation products such as alcohols (e.g., ethanol, methanol, and butanol) are produced from starch-containing material. Enzymatic processes using gelatinized or un-gelatinized (i.e., uncooked) starch-containing material are used in industry.

Conventional fermentation product production processes include a separate enzymatic liquefaction step which is often carried out by heating a starch-containing aqueous slurry to between 80-100° C. and adding alpha-amylase to initiate hydrolysis of the gelatinized slurry. The slurry may then be jet-cooked at a temperature between 105-125° C. to complete gelatinization of the slurry, cooled to 60-95° C. and, generally, additional alpha-amylase is added to finalize hydrolysis.

Producing a fermentation product from un-gelatinized starch may be carried out without a liquefaction step. The un-gelatinized starch may be saccharified and fermented in one step at temperatures below the initial genatination temperature.

Starch hydrolysis in a conventional process is very energy consuming due to the different temperature requirements during the various steps.

U.S. Pat. No. 4,316,956 provides a fermentation process for conversion of un-gelatinized granular starch into ethanol.

U.S. Pat. No. 5,756,714 discloses a method of using a phytase during a conventional liquefaction process.

WO 2001/62947 discloses a method of using a phytase during an ethanol fermentation process.

### SUMMARY OF THE INVENTION

It is an object of the present invention is to provide processes for producing fermentation products from un-gelatinized (i.e., uncooked) starch-containing material.

In the first aspect the invention relates to processes for producing fermentation products from starch-containing material comprising:

(a) saccharifying starch-containing material with an alpha-amylase at a temperature below the initial gelatinization temperature of said starch-containing material, and

(b) fermenting using a fermenting organism,

wherein a phytase is present during saccharification step (a) and/or fermentation step (b).

In a preferred embodiment saccharification step (a) and fermentation step (b) is carried out simultaneously (i.e., one step fermentation process). In a preferred embodiment a carbohydrate-source generating enzyme, preferably glucoamylase, and/or protease is also present during step (a) and/or step (b) or simultaneous saccharification and fermentation in combined steps (a) and (b). In another preferred embodiment an alpha-glucosidase is added during steps (a) and/or (b) or simultaneous steps (a) and (b).

### DETAILED DESCRIPTION OF THE INVENTION

### Producing Fermentation Products from Un-Gelatinized (Uncooked) Starch-Containing Materials

In this aspect the invention relates to processes for producing a fermentation product from starch-containing material without gelatinization of the starch-containing material. According to the invention a desired fermentation product, such as ethanol, may be produced without liquefying the aqueous slurry containing the starch-containing material. In one embodiment a process of the invention includes saccharifying (e.g. milled) starch-containing material, e.g., granular starch, below the initial gelatinization temperature in the presence of an alpha-amylase (see “Alpha-Amylase” section below); and further a carbohydrate-source generating enzyme, preferably a glucoamylase, disclosed below in the “Carbohydrate-source generating enzymes” section below, to produce sugars that can be fermented or converted into a desired fermentation product by a suitable fermenting organism. When further adding alpha-glucosidase, especially together with an alpha-amylase and/or protease, the ethanol yield increased even further. Examples of suitable alpha-glucosidases are listed below in the “Alpha-Glucosidase” section.

Firstly, the invention relates to a process for producing a fermentation product from starch-containing material comprising:

(a) saccharifying starch-containing material with an alpha-amylase at a temperature below the initial gelatinization temperature of said starch-containing material, and

(b) fermenting using a fermenting organism,

wherein a phytase is present during saccharification step (a) and/or fermentation step (b).

Steps (a) and (b) of the process of the invention may be carried out sequentially or simultaneously.

The term “below the initial gelatinization temperature” means the lowest temperature at which gelatinization of the starch commences. Starch heated in water begins to gelatinize between about 50° C. and 75° C.; the exact temperature of gelatinization depends on the specific starch, but can readily be determined by the skilled artisan. Thus, the initial gelatinization temperature may vary according to the plant species, to the particular variety of the plant species as well as with the growth conditions. In the context of this invention the initial gelatinization temperature of a given starch-containing material can be defined as the temperature at which birefringence is lost in 5% of the starch granules using the method described by Gorinstein and Lii, 1992, Starch/Stärke 44(12): 461-466.

Before step (a) a slurry of starch-containing material, such as granular starch, having 10-55 wt, % dry solids, preferably 25-40 wt, % dry solids, more preferably 30-35 wt, % dry solids of starch-containing material may be prepared. The slurry may include water and/or process waters, such as thin stillage (backset), scrubber water, evaporator condensate or distillate, side stripper water from distillation, or other fermentation product plant process water. Because the process of the invention is carried out below the gelatinization temperature and thus no significant viscosity increase takes place, high levels of stillage (backset) may be used if desired, in an embodiment the aqueous slurry contains from about 1 to about 70 vol. % stillage (backset), preferably 15-60 vol. % stillage (backset), especially from about 30 to 50 vol. % stillage (backset).

The starch-containing material may be prepared by reducing the particle size, preferably by dry or wet miffing, to 0.05 to 3.0 mm, preferably 0.1-0.5 mm. After being subjected to a process of the invention at least 60%, at least 70%, at least 80%, at least 90%, at least 90%, at least 91%, at least 92%, at least 93%, at least 94%, at least 95%, at least 96%, at least 97%, at least 98%, or preferably at least 99% of the dry solids of the starch-containing material is converted into a soluble starch hydrolysate.

The process of the invention is conducted at a temperature below the initial gelatinization temperature. In one embodiment, the temperature at which step (a) is carried out sequentially is between 30-75° C., preferably between 45-60° C.

In a preferred embodiment steps (a) and (b) are carried out as a simultaneous saccharification and fermentation process (i.e., one-step fermentation process). In such preferred embodiment the process may typically be carried at a temperature between 20° C. and 40° C., such as between 26° C. and 36° C., such as between 30° C. and 34° C., such as around 32° C. The optimal temperature depends on the fermentation organism.

In an embodiment steps (a) and (b) are carried out simultaneously (i.e., one-step fermentation) so that the sugar level, such as glucose level, is kept at a low level such as below 6 wt. %, preferably below about 3 wt. %, preferably below about 2 wt. %, more preferred below about 1 wt. %, even more preferred below about 0.5 wt. %, or even more preferred 0.25 wt, %, such as below about 0.1 wt, %. Such low levels of sugar can be accomplished by simply employing adjusted quantities of enzyme and fermenting organism

A skilled person in the art can easily determine which quantities of enzyme and fermenting organism to use. The employed quantities of enzyme and fermenting organism may also be selected to maintain low concentrations of maltose in the fermentation broth. For instance, the maltose level may be kept below about 0.5 wt % or below about 0.2 wt. %.

The process of the invention may be carried out at a pH in the range between 3-7, preferably from pH 3-6, or more preferably from pH 4-5

### Starch-Containing Materials

Any suitable starch-containing starting material, including granular starch, may be used according to the present invention. As indicated above the starch-containing material is un-gelatinized (i.e., uncooked) during the process of the invention.

The actual starting material is generally selected based on the desired fermentation product. Examples of starch-containing starting materials suitable for use in a process of present invention, include tubers, roots, stems, whole grains, corns, cobs, wheat, bailey, rye, milo, sago, cassava, tapioca, sorghum, rice peas, beans, or sweet potatoes, or mixtures thereof, or cereals, sugar-containing raw materials, such as molasses, fruit materials, sugar cane or sugar beet, potatoes, and cellulose-containing materials, such as wood or plant residues, or mixtures thereof. Contemplated are both waxy and non-waxy types of corn and barley.

The term “granular starch” means raw uncooked starch, i.e., starch in its natural form found in cereal, tubers or grains. Starch is formed within plant cells as tiny granules insoluble in water. When put in cold water, the starch granules may absorb a small amount of the liquid and swell. At temperatures up to 50° C. to 75° C. the swelling may be reversible. However, with higher temperatures an irreversible swelling called “gelatinization” begins. Granular starch to be processed may be a highly refined starch quality, preferably at least 90%, at least 95%, at least 97% or at least 99.5% pure or it may be a more crude starch containing material comprising, e.g. milled whole grains including non-starch fractions such as germ residues and fibers.

### Fractionation of Starch-Containing Material

In an embodiment the starch-containing material is fractionated into one or more components, including fiber, germ, and a mixture of starch and protein (endosperm). Fractionation may according to the invention be done using any suitable technology or apparatus. For instance, Satake Corporation (Japan) has manufactured a system suitable for fractionation of plant material such as corn.

The germ and fiber components may be fractionated from the remaining portion of the endosperm. In an embodiment of the invention the starch-containing material is plant endosperm, preferably corn endosperm. Further, the endosperm may be reduced in particle size and combined with the larger pieces of the fractionated germ and fiber components for fermentation.

Fractionation can be accomplished, e.g., using the apparatus disclosed in U.S. Application Publication No. 2004/0043117 (hereby incorporated by reference). Suitable methods and apparatus for fractionation include a sieve, sieving and elutriation. Suitable apparatus also include friction mills, such as rice or grain polishing mills (e.g., those manufactured by Satake Corporation (Japan), Kett, or Rapsco, TX (US)).

### Reducing the Particle Size of Starch-Containing Plant Material

The starch-containing material may preferably be reduced in particle size in order to open up the structure and expose more surface area. This may be done by milling. Two milling processes are preferred according to the invention: wet and dry milling. In dry milling whole kernels are milled and used. Wet milling gives a good separation of germ and meal (starch granules and protein) and is often applied at locations where the starch hydrolysate is used in production of syrups. Both dry and wet milling is well known in the art of starch processing and is equally contemplated for the process of the invention. Examples of other contemplated technologies for reducing the particle size of the starch-containing plant material include emulsifying technology and rotary pulsation.

The starch-containing material may be reduced in particle size to between 0.05 to 3.0 mm, or so that at least 30%, preferably at least 50%, more preferably at least 70%, even more preferably at least 90% of the starch-containing material fit through a sieve with a 0.05 to 3.0 mm screen, preferably 0.1-0.5 mm screen.

### Fermentation Products

The term “fermentation product” means a product produced by a process including a fermentation step using a fermenting organism. Fermentation products contemplated according to the invention include alcohols (e.g., ethanol, methanol, butanol); organic acids (e.g., citric acid, acetic acid, itaconic acid, lactic acid, gluconic acid); ketones (e.g., acetone); amino acids (e.g., glutamic acid); gases (e.g., H₂ and CO₂): antibiotics (e.g., penicillin and tetracycline); enzymes; vitamins (e.g., riboflavin, B₁₂, beta-carotene); and hormones. In a preferred embodiment the fermentation product is ethanol, e.g., fuel ethanol: drinking ethanol, i.e., potable neutral spirits; or industrial ethanol or products used in the consumable alcohol industry (e.g., beer and wine), dairy industry (e.g., fermented dairy products), leather industry and tobacco industry. Preferred beer types comprise ales, stouts, porters, lagers, bitters, malt liquors, happoushu, high-alcohol beer, low-alcohol beer, low-calorie beer or light beer. Preferred fermentation processes used include alcohol fermentation processes, as are well known in the art. Preferred fermentation processes are anaerobic fermentation processes, as are well known in the art.

### Fermenting Organisms

“Fermenting organism” refers to any organism, including bacterial and fungal organisms, suitable for use in a fermentation process and capable of producing desired a fermentation product. Especially suitable fermenting organisms are able to ferment, i.e., convert, sugars, such as glucose or maltose, directly or indirectly into the desired fermentation product. Examples of fermenting organisms include fungal organisms, such as yeast. Preferred yeast includes strains of Saccharomyces spp., in particular, Saccharomyces cerevisiae. Commercially available yeast include, e.g., RED STAR™/Lesaffre, ETHANOL RED™ (available from Red Star/Lesaffre, USA) FALI (available from Fleischmann's Yeast, a division of Burns Philp Food Inc., USA), SUPERSTART (available from Alltech), GERT STRAND (available from Geri Strand AB, Sweden) and FERMIOL™ (available from DSM Specialties).

### Enzymes

### Phytase

The phytase used according to the invention may be any enzyme capable of effecting the liberation of inorganic phosphate from phytic acid (myo-inositol hexakisphosphate) or from any salt thereof (phytates). Phytases can be classified according to their specificity in the initial hydrolysis step, viz. according to which phosphate-ester group is hydrolyzed first. A phytase to be used in a process of the invention may have any specificity, e.g., may be a 3-phytase (EC 3.1.3.8), a 6-phytase (EC 3.1.3.26) or a 5-phytase (no EC number).

In a preferred embodiment the phytase has a temperature optimum in the range from 25-70° C., preferably 28-50° C., especially 30-40° C.

In another embodiment the phytase has a temperature optimum above 50° C., such as in the range from 50-70° C.

The phytase may be derived from plants or microorganisms, such as bacteria or fungi, e.g., yeast or filamentous fungi.

The plant phytase may be from wheat-bran, maize, soy bean or lily pollen. Suitable plant phytases are described in Thomlinson et al., 1962, Biochemistry 1: 166-171; Barrientos et al., 1994, Plant. Physiol. 106: 1489-1495; WO 98/05785; and WO 98/20139.

A bacterial phytase may be from genus Bacillus, Citrobacter, Pseudomonas or Escherichia, specifically the species B. subtilis or E. coli. Suitable bacterial phytases are described in Paver and Jagannathan, 1982, Journal of Bacteriology 151: 1102-1108; Cosgrove, 1970, Australian Journal of Biological Sciences 23: 1207-1220; Greiner et al., 1993, Arch. Biochem. Biophys. 303: 107-113; WO 98/06856: WO 97/33976; and WO 97/48812.

A yeast phytase or myo-inositol monophosphatase may be derived from genus Saccharomyces or Schwanniomyces, specifically species Saccharomyces cerevisiae or Schwanniomyces occidentalis, The former enzyme has been described as a Suitable yeast phytases are described in Nayini et al., 1984, Lebensmittel Wissenschaft und Technologie 17: 24-26; Wodzinski et al., Adv. Appl. Microbiol. 42: 263-303; and AU-A-24840/95;

Phytases from filamentous fungi may be derived from the fungal phylum of Ascomycota (ascomycetes) or the phylum Basidiomycota, e.g., the genus Aspergillus, Thermomyces (also called Humicola), Myceliophthora, Manascus, Penicillium, Peniophora, Agrocybe, Paxillus, or Trarnetes, specifically the species Aspergillus terreus, Aspergillus niger, Aspergillus niger var. awamori, Aspergillus ficuum, Aspergillus fumigatus, Aspergillus oryzae, T. lanuginosus (also known as H. lanuginosa), Myceliophthora thermophila, Peniophora lycii, Agrocybe pediades, Manascus anka, Paxillus involtus, or Trametes pubescens. Suitable fungal phytases are described in Yamada et al., 1986, Agric. Biol. Chem. 322: 1275-1282; Piddington et al. 1993, Gene 133: 55-62; EP 684 313; EP 420 358; WO 98/028408; WO 98/28409; JP 7-67635; WO 98/44125; WO 97/38096; and WO 98/13480.

In a preferred embodiment the phytase is the phytase derived from Peniophora lycii disclosed in WO 98/028408.

Modified phytases or phytase variants are obtainable by methods known in the art, in particular by the methods disclosed in EP 897 010; EP 897 985; WO 99/49022; WO 99/48380.

Commercially available phytases contemplated according to the invention include BIO-FEED PHYTASE™, RONOZYME™ P, PHYTASE NOVO™ CT or L (all from Novozymes), or NATUPHOS™ NG 5000 (from DSM); and OPTIPHOS™, PHYZYME™ XP TPT (Danisco, DK).

The dosage of phytase may be in the range 0.001-10 FYT/g DS, particularly 0.01-1 FYT/g DS. A preferred suitable dosage of the phytase is in the range from 0.001-10 mg enzyme protein/g DS, preferably 0.01-1 mg enzyme protein/g DS.

### Protease

A protease may advantageously be present during a process of the invention in addition to phytase. The protease may be of any origin.

The presence of protease may increase the FAN (Free Amino Nitrogen) level and increases the rate of metabolism of the fermenting organism, such as yeast, and may further give higher fermentation efficiency. As used herein, peptidases, proteolytic preparations and protein degrading enzymes are referred to as proteases. In a preferred embodiment the protease is an endo-protease and/or an exo-protease.

Suitable proteases may be of fungal, bacterial, including filamentous fungi and yeast, or plant origin.

In a preferred embodiment the protease is an acid protease, a protease characterized by the ability to hydrolyze proteins under acidic conditions below pH 7, e.g., at pHs in the range from 2 to 7. In an embodiment the acid protease has an optimum pH in the range from 2.5 and 3.5 (determined on high nitrogen casein substrate at 0.7% w/v at 37° C.) and a temperature optimum between 5 to 50° C. at an enzyme concentration of 10 mg/mL at 30′C for one hour in 0.1 M piperazine/acetate/glycine buffer).

In another embodiment the protease is an alkaline protease, i.e., a protease characterized by the ability to hydrolyze proteins under alkaline conditions above pH 7, e.g., at a pH between 7 and 11. In an embodiment the alkaline protease is derived from a strain of Bacillus; preferably Bacillus licheniformis. In an embodiment the alkaline protease has an optimum temperature in the range from 7 and 11 and a temperature optimum around 70° C. determined at pH 9.

In another embodiment the protease is a neutral protease, i.e. a protease characterized by the ability to hydrolyze proteins under conditions between pH 5 and 8. In an embodiment the alkaline protease is derived from a strain of Bacillus, preferably Bacillus amyloliquefaciens. In an embodiment the alkaline protease has an optimum pH in the range between 7 and 11 (determined at 25° C., 10 minutes reaction time with an enzyme concentration of 0.01-0.2 AU/L) and a temperature optimum between 50° C. and 70° C. (determined at pH 8.5, 10 minutes reaction time and 0.03-0.3 AU/L enzyme concentration.

In an embodiment the protease is a metalloprotease. In a preferred embodiment the protease is derived from a strain of the genus Thermoascus, preferably a strain of Thermoascus aurantiacus, especially Thermoaccus aurantiacus CGMCC No. 0670 having the sequence shown in the mature part of SEQ ID NO: 2 in WO 03/048353 hereby incorporated by reference. The Thermoaccus aurantiacus protease is active from 20-90° C., with an optimum temperature around 70° C. Further, the enzyme is activity between pH 5-10 with an optimum around pH 6.

Suitable plant proteases may be derived from barley.

Suitable bacterial proteases include Bacillus proteases derived from Bacillus amyloliquefaciens and Bacillus licheniformis. Suitable filamentous bacterial proteases may be derived from a strain of Nocardiopsis, preferably Nocardiopsis prasina NRRL 18262 protease (or Nocardiopsis sp. 10R) and Nocardiopsis dassonavilla NRRL 18133 (Nocardiopsis dassonavilla M58-1) both described in WO 88/03947 (Novozymes).

Suitable acid fungal proteases include fungal proteases derived from Aspergillus, Mucor, Rhizomucor, Rhizopus, Candida, Coriolus, Endothia, Enthomophtra, Irpex, Penicillium, Sclerotium, Thermoaccus, and Torulopsis. Especially contemplated are proteases derived from Aspergillus niger (see, e.g. Koaze et al., 1964, Agr. Biol. Chem. Japan 28: 216), Aspergillus saitoi (see, e.g., Yoshida, 1954, J. Agr. Chem. Soc. Japan 28: 66), Aspergillus awamori (Hayashida et al., 1977, Agric. Biol. Chem. 42(5): 927-933, Aspergillus aculeatus (WO 95/02044), or Aspergillus oryzae; proteases from Mucor pusillus or Mucor miehei disclosed in U.S. Pat. Nos. 3,988,207 and 4,357.357: and Rhizomucor mehei or Rhizomucor pusillus disclosed in, e.g., WO 94/24880 (hereby incorporated by reference).

In an embodiment the protease is a protease preparation derived from a strain of Aspergillus, such as Aspergillus oryzae. In another embodiment the protease is derived from a strain of Rhizomucor, preferably Rhizomucor miehei. In another contemplated embodiment the protease is a protease preparation, preferably a mixture of a proteolytic preparation derived from a strain of Aspergillus, such as Aspergillus oryzae, and a protease derived from a strain of Rhizomucor, preferably Rhizomucor miehei.

Aspartic acid proteases are described in, for example, Handbook of Proteolytic Enzymes, Edited by A. J. Barrett, N. D. Rawlings and J. F. Messner, Aca-demic Press, San Diego, 1998, Chapter 270). Suitable examples of aspartic acid protease include, e.g., those disclosed in Berka et al., 1990, Gene 96: 313); Berka et al., 1993, Gene 125: 195-198: and Gomi et al., 1993, Biosci. Biotech. Biochem. 57: 1095-1100, which are hereby incorporated by reference.

Commercially available products include ALCALASE®, ESPERASE™, FLAVOURZYME™, PROMIX™, NEUTRASE®, RENNILASE®, NOVOZYM™ FM 2.0L, and NOVOZYM™ 50006 (available from Novozymes A/S, Denmark) and GC106™ and SPEZYME™ FAN from Genencor Int., Inc., USA.

The protease may be present in an amount of 0.0001-1 mg enzyme protein per g DS, preferably 0.001 to 0.1 mg enzyme protein per g DS. Alternatively, the protease may be present in an amount of 0.0001 to 1 LAPU/g DS, preferably 0.001 to 0.1 LAPU/g DS and/or 0.0001 to 1 mAU-RH/g DS, preferably 0.001 to 0.1 mAU-RH/g DS.

### Alpha-Amylase

The aloha-amylase used in a process of the invention may be any alpha-amylase. Preferred are alpha-amylases of fungal or bacterial origin.

In a preferred embodiment the alpha-amylase is an acid alpha-amylase. The acid alpha-amylase may be of fungal or bacterial origin. The term “acid alpha-amylase” means an alpha-amylase (E.C. 3.2.1.1) which added in an effective amount has activity optimum at a pH in the range of 2 to 7, preferably from 3 to 6, or more preferably from 3.5-5.5.

### Bacterial Alpha-Amylases

According to the invention a bacterial alpha-amylase may preferably be derived from the genus Bacillus.

In a preferred embodiment the Bacillus alpha-amylase is derived from a strain of B. licheniformis, B. amyloliquefaciens, B. subtilis or B. stearothermophilus, but may also be derived from other Bacillus sp. Specific examples of contemplated alpha-amylases include the Bacillus licheniformis alpha-amylase (BLA) shown in SEQ ID NO: 4 in WO 99/19467, the Bacillus amyloliquefaciens alpha-amylase (BAN) shown in SEQ ID NO: 5 in WO 99/19467, and the Bacillus stearothermophilus alpha-amylase (BSG) shown in SEQ ID NO: 3 in WO 99/19467. In an embodiment of the invention the alpha-amylase is an enzyme having a degree of identity of at least 60%, preferably at least 70%, more preferred at least 80%, even more preferred at least 90%, such as at least 95%, at least 96%, at least 97%, at least 98% or at least 99% identity to any of the sequences shown as SEQ ID NOS: 1, 2, 3, 4, or 5, respectively, in WO 99/19467.

The Bacillus alpha-amylase may also be a variant and/or hybrid, especially one described in any of WO 96/23873, WO 96/23874, WO 97/41213, WO 99/19467, WO 00/60059, and WO 02/10355 (all documents hereby incorporated by reference). Specifically contemplated alpha-amylase variants are disclosed in U.S. Pat. Nos. 6,093,562, 6,297,038 or 6,187,576 (hereby incorporated by reference) and include Bacillus stearothermophilus alpha-amylase (BSG alpha-amylase) variants having a deletion of one or two amino acid in position 179 to 182, preferably a double deletion disclosed in WO 1996/023873—see e.g., page 20, lines 1-10 (hereby incorporated by reference), preferably corresponding to delta(181-182) compared to the wild-type BSG alpha-amylase amino acid sequence set forth in SEQ ID NO: 3 disclosed in WO 99/19467 or deletion of amino acids 179 and 180 using SEQ ID NO: 3 in WO 99/19467 for numbering (which reference is hereby incorporated by reference). Even more preferred are Bacillus alpha-amylases, especially Bacillus stearothermophilus alpha-amylase, which have a double deletion corresponding to delta(181-182) and further comprise a N193F substitution (also denoted I181*+G182*+N193F) compared to the wild-type BSG alpha-amylase amino acid sequence set forth in SEQ ID NO: 3 disclosed in WO 99/19467.

The aloha-amylase may also be a maltogenic alpha-amylase. A “maltogenic alpha-amylase” (glucan 1,4-alpha-maltohydrolase, E.C. 3.2.1.133) is able to hydrolyze amylose and amylopectin to maltose in the alpha-configuration. A maltogenic alpha-amylase from Bacillus stearothermophilus strain NCIB 11837 is commercially available from Novozymes A/S, Denmark. The maltogenic alpha-amylase is described in U.S. Pat. Nos. 4,598,048, 4,604,355 and 6,162,628, which are hereby incorporated by reference.

### Bacterial Hybrid Alpha-Amylases

A hybrid alpha-amylase specifically contemplated comprises 445 C-terminal amino acid residues of the Bacillus licheniformis alpha-amylase (shown as SEQ ID NO: 4 in WO 99/19467) and the 37 N-terminal amino acid residues of the alpha-amylase derived from Bacillus amyloliquefaciens (shown as SEQ ID NO: 3 in WO 99/194676), with one or more, especially all, of the following substitutions:

G48A+T49I+G107A+H156Y+A181T+N190F+I201F+A209V+Q264S (using the Bacillus licheniformis numbering). Also preferred are variants having one or more of the following mutations (or corresponding mutations in other Bacillus alpha-amylase backbones): H154Y, A181T, N190F, A209V and 0264S and/or deletion of two residues between positions 176 and 179, preferably deletion of E178 and G179 (using the SEQ ID NO: 5 numbering of WO 99/19467).

The bacterial alpha-amylase may be added in amounts well known in the art.

### Fungal Alpha-Amylases

Acid fungal alpha-amylases include acid alpha-amylases derived from a strain of the genus Aspergillus, such as Aspergillus oryzae, Aspergillus niger, and Aspergillus kawachii.

A preferred acid fungal alpha-amylase is a Fungamyl-like alpha-amylase which is preferably derived from a strain of Aspergillus oryzae. In the present disclosure, the term “Fungamyl-like alpha-amylase” indicates an alpha-amylase which exhibits a high identity, i.e., more than 70%, more than 75%, more than 80%, more than 85% more than 90%, more than 95%, more than 96%, more than 97%, more than 98%, more than 99% or even 100% identity to the mature part of the amino acid sequence shown in SEQ ID NO: 10 in WO 96/23874.

Another preferred acid alpha-amylase is derived from a strain Aspergillus niger. In a preferred embodiment the acid fungal alpha-amylase is the one from A. niger disclosed as “AMYA_ASPNG” in the Swiss-prot/TeEMBL database under the primary accession no. P56271 and described in more detail in WO 89/01969 (Example 3). The acid Aspergillus niger acid alpha-amylase is also shown as SEQ ID NO: 1 in WO 2004/080923 (Novozymes) which is hereby incorporated by reference. Also variants of said acid fungal amylase having at least 70% identity, such as at least 80% or even at least 90% identity, such as at least 95%, at least 96%, at least 97%, at least 98%, or at least 99% identity to SEQ ID NO: 1 in WO 2004/080923 are contemplated. A suitable commercially available acid fungal alpha-amylase derived from Aspergillus niger is SP288 (available from Novozymes A/S, Denmark).

In a preferred embodiment the alpha-amylase is derived from Aspergillus kawachii and disclosed by Kaneko et al., 1996, J. Ferment. Bioeng. 81: 292-298 “Molecular-cloning and determination of the nucleotide-sequence of a gene encoding an acid-stable alpha-amylase from Aspergillus kawachii”; and further as EMBL:#AB008370.

The fungal acid alpha-amylase may also be a wild-type enzyme comprising a carbohydrate-binding module (CBM) and an alpha-amylase catalytic domain (i.e., a non-hybrid), or a variant thereof. In an embodiment the wild-type acid alpha-amylase is derived from a strain of Aspergillus kawachii.

### Fungal Hybrid Alpha-Amylases

In a preferred embodiment the fungal acid alpha-amylase is a hybrid alpha-amylase. Preferred examples of fungal hybrid alpha-amylases include the ones disclosed in WO 2005/003311 or U.S. Application Publication No. 2005/0054071 (Novozymes) or U.S. application No. 60/638,614 (Novozymes) which is hereby incorporated by reference. A hybrid alpha-amylase may comprise an alpha-amylase catalytic domain (CD) and a carbohydrate-binding domain/module (CBM) and optional a linker.

Specific examples of contemplated hybrid alpha-amylases include those disclosed in U.S. application No. 60/638,614 including Fungamyl variant with catalytic domain JA118 and Athelia rolfsii SBD (SEQ ID NO: 100 in U.S. application No. 60/638,614), Rhizomucor pusillus alpha-amylase with Athelia rolfsii AMG linker and SBD (SEQ ID NO: 101 in U.S. application No. 60/638,614) and Meripilus giganteus alpha-amylase with Athelia rolfsii glucoamylase linker and SBD (SEQ ID NO: 102 in U.S. application no, 60/638,614).

Other specific examples of contemplated hybrid alpha-amylases include those disclosed in U.S. Application Publication No. 2005/0054071, including those disclosed in Table 3 on page 15, such as Aspergillus niger alpha-amylase with Aspergillus kawachii linker and starch binding domain.

### Commercial Alpha-Amylase Products

Commercial compositions comprising alpha-amylase include MYCOLASE™ from DSM (Gist Brocades), BAN™, TERMAMYL™ SC, FUNGAMYL™, LIQUOZYME™ X and SAN™ SUPER, SAN™ EXTRA L (Novozymes A/S) and CLARASE™ L-40,000, DEX-LO™, SPEZYME™ FRED, SPEZYME™ AA, SPEZYME™ HPA and SPEZYME™ DELTA AA (Genencor Int.), and the acid fungal alpha-amylase sold under the trade name SP288 (available from Novozymes A/S, Denmark).

An acid alpha-amylases may according to the invention be added in an amount of 0.1 to 10 AFAU/g DS, preferably 0.10 to 5 AFAU/g DS, especially 0.3 to 2 AFAU/g DS or 0.001 to 1 FAU-F/g DS, preferably 0.01 to 1 FAU-F/g DS.

When measured in KNU units the alpha-amylase activity is preferably present in an amount of 0.0005-5 KNU per g DS, preferably 0.001-1 KNU per g DS, such as around 0.050 KNU per g DS.

### Carbohydrate-Source Generating Enzyme

The term “carbohydrate-source generating enzyme” includes glucoamylase (being glucose generators), beta-amylase and maltogenic amylase (being maltose generators). A carbohydrate-source generating enzyme is capable of producing a carbohydrate that can be used as an energy-source by the fermenting organism(s) in question, for instance, when used in a process of the invention for producing a fermentation product, such as ethanol. The generated carbohydrate may be converted directly or indirectly to the desired fermentation product, preferably ethanol. According to the invention a combination or mixture of carbohydrate-source generating enzyme and alpha-amylase may be used in a process of the invention. Especially contemplated combinations or mixtures are include one or more glucoamylases as disclosed below in the “Glucoamylases”-section and an alpha-amylase as defined in the Alpha-Amylase”-section below. The ratio between alpha-amylase activity (AFAU) per glucoamylase activity (AGU) (AFAU per AGU) may in an embodiment of the invention be at least 0.1, in particular at least 0.16, such as in the range from 0.12 to 0.50 or more. In a preferred embodiment the alpha-amylase and glucoamylase is added in a ratio of between 0.1 and 100 AGU/FAU-F, preferably 2 and 50 AGU/FAU-F, especially between 10 and 40 AGU/FAU-F when steps (a) and (b) are carried out simultaneously.

### Glucoamylases

A glucoamylase used according to the invention may be derived from any suitable source, e.g., derived from a microorganism or a plant. Preferred glucoamylases are of fungal or bacterial origin, selected from the group consisting of Aspergillus glucoamylases, in particular A. niger G1 or G2 glucoamylase (Boel et al., 1984, EMBO J. 3 (5): 1097-1102), or variants thereof, such as those disclosed in WO 92/00381, WO 00/04136 and WO 01/04273 (from Novozymes, Denmark); the A. awamori glucoamylase disclosed in WO 84/02921, A. oryzae glucoamylase (1991, Agric. Biol. Chem. 55 (4): 941-949), or variants or fragments thereof. Other Aspergillus glucoamylase variants include variants with enhanced thermal stability: G137A and G139A (Chen et al. 1996, Prot. Eng. 9: 499-505); D257E and D293E/0 (Chen et al., 1995, Prot. Eng. 8: 575-582); N182 (Chen et al. 1994, Biochem J. 301: 275-281); disulphide bonds, A246C (Fierobe et al., 1996, Biochemistry 35: 8698-8704; and introduction of Pro residues in position A435 and 5436 (Li et al., 1997, Protein Eng. 10: 1199-1204).

Other glucoamylases include Athelia rolfsii (previously denoted Corticium rolfsii) glucoamylase (see U.S. Pat. No. 4,727,026 and Nagasaka et al., 1998, “Purification and properties of the raw-starch-degrading glucoamylases from Corticium rolfsii, App. Microbiol. Biotechnol. 50: 323-330), Talaromyces glucoamylases, in particular derived from Talaromyces emersonii WO 99/28448), Talaromyces leycettanus (U.S. Pat. No. Re. 32,153), Talaromyces duponti, Talaromyces thermophilus (U.S. Pat. No. 4,587,215).

Bacterial glucoamylases contemplated include glucoamylases from the genus Clostridium, in particular C. thermoamylolyticum (EP 135 138), and C. thermohydrosulfuricum WO 86/01831).

Also hybrid glucoamylase are contemplated according to the invention. Examples the hybrid glucoamylases disclosed in WO 2005/045018. Specific examples include the hybrid glucoamylase disclosed in Table 1 and 4 of Example 1 (which hybrids are hereby incorporated by reference).

Preferred glucoamylases are the glucoamylase is selected from the group consisting of glucoamylases derived from the genus Aspergillus, preferably a strain of Aspergillus niger, Aspergillus oryzae, Aspergillus awamori; or the genus Athelia, preferably a strain of Athelia rolfsii: the genus Talaromyces, preferably a strain the Talaromyces emersonii; or the genus Rhizopus, such as a strain of Rhizopus nivius; or of the genus Humicola, preferably a strain of Humicola grisea var. thermoidea; or a strain of the genus Pachykytospora, preferably Pachyhytospora papyracea; a strain of the genus Leocopaxillus, preferably Leucopaxillus giganteus; or a strain of the genus Trametes, preferably a strain of Trametes cingulata (disclosed in WO 2006/069289 which is hereby incorporated by reference).

The glucoamylase may also be a hydrid glucoamylase. Examples of such hybrids can be found in WO 2005/045018 which is incorporated by reference.

Glucoamylases may in an embodiment be added in an amount of 0.001 to 10 AGU/g DS, preferably from 0.01 to 5 AGU/g DS, especially 0.1 to 1 AGU/g DS.

### Beta-Amylase

A beta-amylase (E.C 3.2.1.2) is the name traditionally given to exo-acting maltogenic amylases, which catalyze the hydrolysis of 1,4-alpha-glucosidic linkages in amylose, amylopectin and related glucose polymers. Maltose units are successively removed from the non-reducing chain ends in a step-wise manner until the mole-cule is degraded or, in the case of amylopectin, until a branch point is reached. The maltose re-leased has the beta anomeric configuration, hence the name beta-amylase.

Beta-amylases have been isolated from various plants and microorganisms (Fogarty and Kelly, 1979, Progress in Industrial Microbiology 15: 112-115). These beta-amylases are characterized by having optimum temperatures in the range from 40° C. to 65° C. and optimum pH in the range from 4.5 to 7. A commercially available beta-amylase from barley is NOVOZYM™ WBA from Novozymes A/S, Denmark and SPEZYME™ BBA 1500 from Genencor Int., USA.

### Maltogenic Amylase

The amylase may also be a maltogenic alpha-amylase, A “maltogenic alpha amylase” (glucan 1,4-alpha-maltohydrolase, E.C. 3.2.1.133) is able to hydrolyze amylose and amylopectin to maltose in the alpha-configuration. A maltogenic amylase from Bacillus stearothermophilus strain NCIB 11837 is commercially available from Novozymes A/S. Maltogenic alpha-amylases are described in U.S. Pat. Nos. 4,598,048, 4,604,355 and 6,162,628, which are hereby incorporated by reference.

The maltogenic amylase may in a preferred embodiment be added in an amount of 0.05-5 mg total protein/gram DS or 0.05-5 MANU/g DS.

### Alpha-Glucosidase

According to the invention an alpha-glucosidase may be added. Any alpha-glucosidase (including enzymes classified as EC 3.2.1.20 or EC 3.2.1.48) may be used according to the invention. Examples of alpha-glucosidases contemplated according to the invention include those derived from microorganisms, such as bacteria and fungi, including yeast and filamentous fungi, Actinomycetes, and plants.

In an embodiment the alpha-glucosidase is an acid alpha-glucosidase. This means that the pH optimum is below 7.0, preferably between pH 3-7.

In a preferred embodiment the alpha-glucosidase is stable in the presence of the fermentation product in question at concentrations below 10 vol. %, preferably below 12 vol. %, more preferably below 15 vol. %, more preferably below 18 vol. %, more preferably below 20 vol, %, more preferably below 25 vol. % fermentation product. In a specific embodiment the alpha-glucosidase is stable in the presence of ethanol, preferably at concentrations below 10 vol. %, preferably 12 vol. %, more preferably below 15 vol. %, more preferably below 18 vol, %, even more preferably below 20 vol. %, even more preferably below 25 vol. % ethanol. Ethanol stability means that the relative activity is above 50%, preferably above 70%, more preferably above 90% after 10 minutes, preferably after 30 minutes, more preferably after 60 minutes incubation at 30-40° C., preferably at around 37′C at process conditions.

Bacterial alpha-glucosidases include those derived from a strain of the genus Bacillus, such as a strain of Bacillus stearothermophilus. A commercial Bacillus stearothermophilus alpha-glucosidase is available from Sigma (Sigma cat. No. G3651).

Fungal alpha-glucosidases include those derived from yeast or filamentous fungi. Examples of alpha-glucosidases derived from yeast include those derived from a strain of Candida sp, such as Candida edax, preferably CBD 6451, or from a strain of Saccharomyces, preferably Saccharomyces cerevisae. Other alpha-glucosidases derived from yeast include those derived from Pichia sp., such as Pichia amylophila, Pichia missisipiensis, Pichia wiherhamii and Pichiarhadanensis.

Alpha-glucosidases derived from filamentous fungi, include those from the genus Aspergillus, Fusarium, Mucor, and Penicillium.

Examples of alpha-glucosidases from a strain of Aspergillus include those derived from Aspergillus nidulans (Kato et al., 2002, Appl. Environ Microbial. 68: 1250-1256), Aspergillus fumigatus (Rudick and Elbein, 1974, Archives of Biochemistry and Biophysics 161: 281-290), Aspergillus flavus (Olutiola, 1981, Mycologia 73: 1130), Aspergillus nidulans (Kato et al., 2002, Appl. Environ, Microbial. 68: 1250-1256), Aspergillus niger (Rudick at al., 1979. Archives of Biochemistry and Biophysics 193: 509 and Nakamura at al., 1997, J. Biotechnol. 53, 75-84). Aspergillus oryzae (Minetoki at al., 1995, Biosci. Biotech. Biochem. 59, 1516-1521; Leibowitz and Mechlinski, 1926, Hoppe-Seyler's Zeitschrift für Physiologische Chemie 154: 64) and Aspergillus fumigatus (U.S. Application Publication No. 2006/0008879). Known alpha-glucosidases also include those derived from a strain of Rhizobium sp. (Berthelot at al. 1999, Appl. Environ Microbial, 65: 2907-2911), Mucor javanicus (Yamasaki at al, 1978. Berichte des Ohara Instituts für Landwirtschaftliche Biologie 17: 123), Mucor racemosus (Yamasaki at al., 1977, Agricultural and Biological Chemistry 41: 1553), Mucor rouxii (Flores-Carreon and Ruiz-Herrera, 1972, Blochemica et Biophysica Acta 258: 496), Penicillium pupuragenum (Yamasaki et al., 1976, Agricultural and Biological Chemistry 40: 669), and Penicillium oxalicum (Yamasaki at al., 1977, Agricultural and Biological Chemistry 41: 1451) and Fusarium venenatum (U.S. Application Publication No. 2006/0156437).

In a preferred embodiment the fungal alpha-glucosidase is derived from a strain of the genus Aspergillus, including A. nidulans, A. niger, A. oryzae and A. fumigatus.

In a preferred embodiment the alpha-glucosidase is a plant alpha-glucosidase. The plant alpha-glucosidase may be derived from any plant material, preferably a plant selected from corn (maize), cobs, wheat, barley, rye, milo, sago, cassava, tapioca, sorghum, rice, peas, or beans, sweet potatoes, or a mixture thereof. In a preferred embodiment the alpha-glucosidase is derived from corn

Contemplated are also alpha-glucosidases which exhibit a high identity to any of above mention alpha-glucosidases, i.e., more than 70%, more than 75%, more than 80%, more than 85% more than 90%, more than 95%, more than 96%, more than 97%, more than 98%, more than 99% or even 100% identity to the mature enzyme sequences.

In an embodiment from 0.01 to 10 AGU/g DS, preferably 0.1 to 5 AGU/g DS of alpha-glucosidase is added.

### Materials & Methods

Phytase: derived from Peniophora lycii and available from Novozymes (Used in all examples)
Glucoamylase: Glucoamylase derived from Trametes cingulata disclosed in SEQ ID NO: 2 in WO 2006/069289 and available from Novozymes A/S (used in all examples).
Alpha-Amylase: Hybrid alpha-amylase consisting of Rhizomucor pusillus alpha-amylase with Aspergillus niger glucoamylase linker and SBD disclosed as V039 in Table 5 in WO 2006/069290 (Novozymes A/S) (used in all examples).
Protease A is a proteolytic enzyme preparation derived from Aspergillus oryzae available from Novozymes A/S, Denmark
Protease B is Mucorpepsin, which is a clan AA-peptidase family A1 enzyme derived from Rhizomucor miehei and produced recombinantly in Aspergillus oryzae. The enzyme is available on request from Novozymes A/S, Denmark.
Alpha-glucosidase F is derived from Aspergillus fumigatus and is disclosed in U.S. Application Publication No, 2006/0008879 (Novozymes)
Alpha-glucosidase N is derived from Aspergillus niger.
Yeast: RED STAR™ available from Red Star/Lesaffre, USA
Backset used in Examples 6 and 7 was obtained from a conventional ethanol production plant of in Iowa, USA.

### Media and Reagents:

Chemicals used as buffers and substrates were commercial products of at least reagent grade.

PDA: 39 g/L Potato Dextrose Agar, 20 g/L agar, 50 ml/L glycerol

### Methods

### Identity

The relatedness between two amino acid sequences or between two nucleotide sequences is described by the parameter “identity”.

For purposes of the present invention, the degree of identity between two amino acid sequences is determined by the Clustal method (Higgins, 1989, GABIOS 5: 151-153) using the LASERGENE™ MEGALIGN™ software (DNASTAR, Inc., Madison, Wis.) with an identity table and the following multiple alignment parameters: Gap penalty of 10 and gap length penalty of 10. Pairwise alignment parameters are Ktuple=1, gap penalty=3, windows=5, and diagonals=5.

For purposes of the present invention, the degree of identity between two nucleotide sequences is determined by the Wilbur-Lipman method (Wilbur and Lipman, 1983, Proceedings of the National Academy of Science USA 80: 726-730) using the LASERGENE™ MEGALIGN™ software (DNASTAR, Inc. Madison, Wis.) with an identity table and the following multiple alignment parameters: Gap penalty of 10 and gap length penalty of 10. Pairwise alignment parameters are Ktuple=3, gap penalty=3, and windows=20.

### Protease Activity

### Protease Assay Method (LAPU)

1 Leucine Amino Peptidase Unit (LAPU) is the amount of enzyme which decomposes 1 microM substrate per minute at the following conditions: 26 mM of L-leucine-p-nitroanilide as substrate, 0.1 M Tris buffer (pH 8.0), 37° C., reaction time of 10 minutes.

LAPU is described in EB-SM-0298.02/01 which is available from Novozymes A/S (Denmark) on request.

### Protease Assay Method—AU(RH)

The proteolytic activity may be determined with denatured hemoglobin as substrate. In the Anson-Hemoglobin method for the determination of proteolytic activity denatured hemoglobin is digested, and the undigested hemoglobin is precipitated with trichloroacetic acid (TCA). The amount of TCA soluble product is determined with phenol reagent, which gives a blue color with tyrosine and tryptophan.

One Anson Unit (AU(RH)) is defined as the amount of enzyme which under standard conditions (i.e., 25° C., pH 5.5 and 10 min. reaction time) digests hemoglobin at an initial rate such that there is liberated per minute an amount of TCA soluble product which gives the same color with phenol reagent as one milliequivalent of tyrosine.

AU(RH) is described in EAL-SM-0350 which is available from Novozymes A/S (Denmark) on request.

### Glucoamylase Activity

Glucoamylase activity may be measured in AGI units or in Glucoamylase Units (AGU).

### Glucoamylase Activity (AGI)

Glucoamylase (equivalent to amyloglucosidase) converts starch into glucose. The amount of glucose is determined here by the glucose oxidase method for the activity determination. The method described in the section 76-11 Starch-Glucoamylase Method with Subsequent Measurement of Glucose with Glucose Oxidase in “Approved methods of the American Association of Cereal Chemists”, Vol. 1-2 AACC, from American Association of Cereal Chemists, 2000; ISBN: 1-891127-12-8.

One glucoamylase unit (AGI) is the quantity of enzyme which will form 1 micro mole of glucose per minute under the standard conditions of the method.

### Standard Conditions/Reaction Conditions:

| Substrate: | Soluble starch, concentration | |
|---|---|---|
| | approx. 16 g dry matter/L. | |
| Buffer: | Acetate, approx. 0.04 M, pH = 4.3 | |
| pH: | 4.3 | |
| Incubation temperature: | 60° | C. |
| Reaction time: | 15 | minutes |
| Termination of the reaction: | NaOH to a concentration of | |
| | approximately 0.2 g/L (pH ~9) | |
| Enzyme concentration: | 0.15-0.55 | AAU/mL. |

The starch should be Lintner starch, which is a thin-boiling starch used in the laboratory as colorimetric indicator. Lintner starch is obtained by dilute hydrochloric acid treatment of native starch so that it retains the ability to color blue with iodine.

### Glucoamylase Activity (AGU)

The Novo Glucoamylase Unit (AGU) is defined as the amount of enzyme, which hydrolyzes 1 micromole maltose per minute under the standard conditions 31° C., pH 4.3, substrate: maltose 23.2 mM, buffer: acetate 0.1 M, reaction time 5 minutes.

An autoanalyzer system may be used. Mutarotase is added to the glucose dehydrogenase reagent so that any alpha-D-glucose present is turned into beta-D-glucose. Glucose dehydrogenase reacts specifically with beta-D-glucose in the reaction mentioned above, forming NADH which is determined using a photometer at 340 nm as a measure of the original glucose concentration.

| AMG incubation: | | |
|---|---|---|
| Substrate: | maltose 23.2 | mM |
| Buffer: | acetate 0.1 | M |
| pH: | 4.30 ± 0.05 | |
| Incubation temperature: | 37° C. ± 1 | |
| Reaction time: | 5 | minutes |
| Enzyme working range: | 0.5-4.0 | AGU/mL |
| Color reaction: | | |
| GlucDH: | 430 | U/L |
| Mutarotase: | 9 | U/L |
| NAD: | 0.21 | mM |
| Buffer: | phosphate 0.12 M; 0.15 M NaCl | |
| pH: | 7.60 ± 0.05 | |
| Incubation temperature: | 37° C. ± 1 | |
| Reaction time: | 5 | minutes |
| Wavelength: | 340 | nm |

A folder (EB-SM-0131.02/01) describing this analytical method in more detail is available on request from Novozymes A/S (Denmark), which folder is hereby incorporated by reference.

### Alpha-Amylase Activity (KNU)

The alpha-amylase activity may be determined using potato starch as substrate. This method is based on the break-down of modified potato starch by the enzyme, and the reaction is followed by mixing samples of the starch/enzyme solution with an iodine solution. Initially, a blackish-blue color is formed, but during the break-down of the starch the blue color gets weaker and gradually turns into a reddish-brown, which is compared to a colored glass standard.

One Kilo Novo alpha amylase Unit (KNU) is defined as the amount of enzyme which, under standard conditions (i.e. at 37° C.+/−0.05; 0.0003 M Ca²⁺; and pH 5.6) dextrinizes 5260 mg starch dry substance Merck Amylum solubile.

A folder EB-SM-0009.02/01 describing this analytical method in more detail is available upon request to Novozymes A/S. Denmark, which folder is hereby included by reference.

### Acid Alpha-Amylase Activity

When used according to the present invention the activity of any acid alpha-amylase may be measured in AFAU (Acid Fungal Alpha-amylase Units). Alternatively activity of acid alpha-amylase may be measured in AAU (Acid Alpha-amylase Units),

### Acid Alpha-Amylase Units (AAU)

The acid alpha-amylase activity can be measured in AAU (Acid Alpha-amylase Units), which is an absolute method. One Acid Amylase Unit (AAU) is the quantity of enzyme converting 1 g of starch (100% of dry matter) per hour under standardized conditions into a product having a transmission at 620 nm after reaction with an iodine solution of known strength equal to the one of a color reference.

### Standard Conditions/Reaction Conditions:

| Substrate: | Soluble starch. Concentration | |
|---|---|---|
| | approx. 20 g DS/L. | |
| Buffer: | Citrate, approx. 0.13 M, pH = 4.2 | |
| Iodine solution: | 40.176 g potassium iodide + | |
| | 0.088 g iodine/L | |
| City water | 15°-20° dH | |
| | (German degree hardness) | |
| pH: | 4.2 | |
| Incubation temperature: | 30° | C. |
| Reaction time: | 11 | minutes |
| Wavelength: | 620 | nm |
| Enzyme concentration: | 0.13-0.19 | AAU/mL |
| Enzyme working range: | 0.13-0.19 | AAU/mL |

The starch should be Lintner starch, which is a thin-boiling starch used in the laboratory as colorimetric indicator. Lintner starch is obtained by dilute hydrochloric acid treatment of native starch so that it retains the ability to color blue with iodine. Further details can be found in EP 140 410, which disclosure is hereby included by reference.

### Determination of FAU-F

FAU(F) Fungal Alpha-Amylase Units (Fungamyl) is measured relative to an enzyme standard of a declared strength.

Reaction conditions

| Temperature | 37° | C. |
|---|---|---|
| pH | 7.15 | |
| Wavelength | 405 | nm |
| Reaction time | 5 | min |
| Measuring time | 2 | min |

A folder (EB-SM-0216.02) describing this standard method in more detail is available on request from Novozymes A/S, Denmark, which folder is hereby included by reference.

Acid alpha-Amylase Activity (AFAU)

Acid alpha-amylase activity may be measured in AFAU (Acid Fungal Alpha-amylase Units), which are determined relative to an enzyme standard. 1 AFAU is defined as the amount of enzyme which degrades 5.260 mg starch dry matter per hour under the below mentioned standard conditions.

Acid alpha-amylase, an endo-alpha-amylase (1,4-alpha-D-glucan-glucanohydrolase, E.C. 3.2.1.1) hydrolyzes alpha-1,4-glucosidic bonds in the inner regions of the starch molecule to form dextrins and oligosaccharides with different chain lengths. The intensity of color formed with iodine is directly proportional to the concentration of starch. Amylase activity is determined using reverse colorimetry as a reduction in the concentration of starch under the specified analytical conditions.

$${STARCH + IODINE}_{λ = 590 nm} {{\to }_{{40}^{*} , pH 2 , 5}}^{ALPHA \text{-} AMYLASE} DEXTRINS + OLIGOSACCHARIDES$$

blue/violet t=23 sec. decoloration

Standard Conditions/Reaction Conditions:

| Substrate: | Soluble starch, approx. 0.17 g/L | |
|---|---|---|
| Buffer: | Citrate, approx. 0.03 M | |
| Iodine (I2): | 0.03 | g/L |
| CaCl₂: | 1.85 | mM |
| pH: | 2.50 ± 0.05 | |
| Incubation temperature: | 40° | C. |
| Reaction time: | 23 | seconds |
| Wavelength: | 590 | nm |
| Enzyme concentration: | 0.025 | AFAU/mL |
| Enzyme working range: | 0.01-0.04 | AFAU/mL |

A folder EB-SM-0259.02/01 describing this analytical method in more detail is available upon request to Novozymes A/S (Denmark), which is hereby incorporated by reference.

### Determination of Maltogenic Amylase Activity (MANU)

One MANU (Maltogenic Amylase Novo Unit) may be defined as the amount of enzyme required to release one micro mole of maltose per minute at a concentration of 10 mg of maltotriose (Sigma M 8378) substrate per ml of 0.1 M citrate buffer, pH 5.0 at 37*C for 30 minutes.

### Phytase Activity (FYT)

Phytase activity is determined as FYI units, one FYI being the amount of enzyme that liberates 1 micromole inorganic orthophosphate per min, under the following conditions: pH 5.5: temperature 37′C; substrate: sodium phytate (C₆H₆O₂₄P₆Na₁₂) at a concentration of 0.0050 mole/l.

### EXAMPLES

### Example 1

All treatments were evaluated via mini-scale fermentations. 410 g of ground yellow dent corn (with an average particle size around 0.5 mm) was added to 590 g tap water. This mixture was supplemented with 3.0 mL 1 g/L penicillin and 1 g of urea. The pH of this slurry was adjusted to 4.5 with 5 N NaOH (initial pH, before adjustment was about 3.8). DS level was determined to be about 35 wt. %. Approximately 5 g of this slurry was added to 10 ml tubes. Each tube was dosed with the appropriate amount of enzyme followed by addition of about 0.21 mL yeast propagate per 5 g slurry. Actual enzyme dosages were based on the exact weight of corn slurry in each tube. Tubes were incubated at 32° C. Nine replicate fermentations of each treatment were run. Fermentation tubes were vortexed by hand at 24, 48 and 70 hours. Three tubes were taken at 24 hours, 48 hours, and 70 hours time point to be analyzed by HPLC. The HPLC sample preparation consisted of immediately stopping the reaction by addition of 50 microliters of 40% H₂SO₄ in each 5 g sample, centrifuging at 3000 rpm and at room temperature for 5 minutes, and then filtering through a 0.45 micrometer filter. Samples were stored at 4° C. prior to analysis. Agilent™ 1100 HPLC system coupled with RI detector was used to determine concentrations of ethanol and sugars. The separation column was Aminex HPX-87H ion exclusion column (300 mm×7.8 mm) from BioRad™. The enzyme dose and ethanol yield are shown in Table below:

| Gluco- | | Proteases | | | | | |
|---|---|---|---|---|---|---|---|
| amylase | Alpha- | A | | Phytase | | | |
| AGU | Amylase | LAPU | B | FYT | Ethanol (% v/v) | | |
| per g | FAU-F | per g | mAU(RH) | per g | 24 | 48 | 70 |
| DS | per g DS | Ds | per g DS | DS | hours | hours | hours |
| 0.4 | 0.065 | — | — | — | 12.79 | 17.74 | 18.63 |
| 0.4 | 0.065 | 0.0042 | 0.0025 | — | 12.78 | 17.81 | 18.67 |
| 0.4 | 0.065 | — | — | 0.0550 | 12.98 | 17.85 | 18.70 |
| 0.4 | 0.065 | — | — | 0.0825 | 12.78 | 17.91 | 18.77 |
| 0.4 | 0.065 | — | — | 0.1100 | 12.94 | 17.98 | 18.76 |
| 0.4 | 0.065 | 0.0042 | 0.0025 | 0.0550 | 12.72 | 17.92 | 18.96 |
| 0.4 | 0.065 | 0.0042 | 0.0025 | 0.0825 | 13.11 | 17.99 | 18.82 |
| 0.4 | 0.065 | 0.0042 | 0.0025 | 0.1100 | 12.91 | 18.10 | 18.97 |

### Example 2

The experiments were performed as described in Example 1. The enzyme dose and ethanol yield are shown in Table below:

| Gluco- | | Proteases | | | | | |
|---|---|---|---|---|---|---|---|
| amylase | Alpha- | A | | Phytase | | | |
| AGU | Amylase | LAPU | B | FYT | Ethanol (% v/v) | | |
| per g | FAU-F | per g | mAU(RH) | per g | 24 | 48 | 70 |
| DS | per g DS | Ds | per g DS | DS | hours | hours | hours |
| 0.4 | 0.065 | — | — | — | 12.79 | 17.74 | 18.63 |
| 0.4 | 0.065 | 0.0021 | 0.00125 | — | 12.91 | 17.74 | 18.62 |
| 0.4 | 0.065 | 0.0042 | 0.00250 | — | 12.78 | 17.81 | 18.67 |
| 0.4 | 0.065 | 0.0063 | 0.00375 | — | 12.87 | 17.97 | 18.81 |
| 0.4 | 0.065 | — | — | 0.0825 | 12.78 | 17.91 | 18.77 |
| 0.4 | 0.065 | 0.0021 | 0.00125 | 0.0825 | 13.07 | 17.94 | 18.86 |
| 0.4 | 0.065 | 0.0042 | 0.00250 | 0.0825 | 13.11 | 17.99 | 18.82 |
| 0.4 | 0.065 | 0.0063 | 0.00375 | 0.0825 | 12.97 | 17.91 | 18.92 |

### Example 3

All treatments were evaluated via mini-scale fermentations. 1230 g of ground endosperm from fractionated yellow dent corn was added to 1770 g tap water. The average particle size of endosperm is around 0.5 mm. This mixture was supplemented with 3.0 ml 1 g/L penicillin and 3 g of urea. The pH of this slurry was adjusted to 4.5 with 5 N NaOH. DS level was determined to be about 33.6 wt. %. Approximately 60 g of this slurry was added to 100 mL bottles. Each bottle was dosed with the appropriate amount of enzyme followed by addition of about 2.5 mL yeast propagate per 60 g slurry. Actual enzyme dosages were based on the exact weight of corn slurry in each bottle. Bottles were incubated at 32° C. Three replicate fermentations of each treatment were run. Fermentation bottles were manually shacked by hand at 24, 48 and 70 hours. Fermentation samples were taken at 24 hours. 48 hours, and 70 hours time point to be analyzed by HPLC. The HPLC sample preparation consisted of immediately stopping the reaction by addition of 50 microliters of 40% H₂SO₄ in each 5 g sample, centrifuging at 3000 rpm and at room temperature for 5 minutes, and then filtering through a 0.45 micrometer filter. Samples were stored at 4° C. prior to analysis. Agilent™ 1100 HPLC system coupled with RI detector was used to determine concentration of ethanol and sugars. The separation column was Aminex HPX-87H ion exclusion column (300 mm×7.8 mm) from BioRad™. The enzyme dose and ethanol yield are shown in Table below:

| | Alpha- | Proteases | | | | | |
|---|---|---|---|---|---|---|---|
| Glucoamylase | Amylase | A | B | Phytase | Ethanol (% v/v) | | |
| AGU | FAU-F | LAPU | mAU(RH) | FYT | 24 | 48 | 70 |
| per g DS | per g DS | per g Ds | per g DS | per g DS | hours | hours | hours |
| 0.4 | 0.065 | — | — | — | 100.77 | 141.00 | 146.45 |
| 0.4 | 0.065 | — | — | 0.11 | 100.91 | 143.53 | 150.58 |
| 0.4 | 0.065 | 0.0042 | 0.0025 | — | 100.00 | 143.00 | 149.44 |
| 0.4 | 0.065 | 0.0042 | 0.0025 | 0.11 | 100.77 | 142.62 | 156.60 |

### Example 4

All treatments were performed as described in Example 3, except that ground corn endosperm was used instead of ground whole corn. The dry solid (DS) was determined to be about 35 wt. %. The enzyme dose and ethanol yield are shown in the Table below:

| | Alpha- | Alpha-Glucosidase | | | | | |
|---|---|---|---|---|---|---|---|
| Glucoamylase | Amylase | N | F | Phytase | Ethanol (g/L) | | |
| AGU | FAU-F | AGU | AGU | FYT | | 48 | 70 |
| per g DS | per g DS | per g Ds | per g DS | per g DS | 24 hours | hours | hours |
| 0.4 | 0.065 | — | — | — | 105.90 | 143.83 | 154.23 |
| 0.4 | 0.065 | 2.1 | — | — | 104.41 | 144.71 | 155.82 |
| 0.4 | 0.065 | — | 1.56 | — | 105.97 | 145.40 | 155.76 |
| 0.4 | 0.065 | — | — | 0.11 | 104.73 | 144.62 | 156.19 |
| 0.4 | 0.065 | 2.1 | — | 0.11 | 108.49 | 145.84 | 157.20 |
| 0.4 | 0.065 | — | 1.56 | 0.11 | 112.67 | 149.07 | 158.23 |

### Example 5

All treatments were evaluated via mini-scale fermentations. 103.55 g of ground yellow dent corn (with an average particle size around 0.5 mm) was added in 146.45 g tap water to total 250 g slurry. This mixture was supplemented with 6 mg/kg corn slurry of penicillin and 1 g/kg corn slurry of urea. The pH of this slurry was adjusted to 4.5 with 5 N NaOH. Dry solid (DS) level was determined to be about 34.42 wt %. Approximately 5 g of this slurry was added to 10 mL tubes. Each tube was dosed with the appropriate amount of enzyme followed by addition of about 0.21 mL yeast propagate per 5 g slurry. Actual enzyme dosages were based on the exact weight of corn slurry in each tube. Tubes were incubated at 32° C. Nine replicate fermentations of each treatment were run. Fermentation tubes were vortexed by hand at 20, 47 and 70 hours. Three tubes were taken at 20 hours, 47 hours, and 70 hours time point to be analyzed by HPLC. The HPLC sample preparation consisted of immediately stopping the reaction by addition of 50 microL of 40% H₂SO₄ in each 5 g sample, centrifuging at 3000 rpm and at room temperature for 5 minutes, and then filtering through a 0.45 micrometer filter. Samples were stored at 4° C. prior to analysis, Agilent™ 1100 HPLC system coupled with RI detector was used to determine concentrations of ethanol and sugars. The separation column was Aminex HPX-87H ion exclusion column (300 mm×7.8 mm) from BioRad™. The enzyme dose and ethanol yield are shown in Table below. As phytase concentration increases, the % (w/v) ethanol yield increases.

| Treat- | | Alpha- | Phytase | | | |
|---|---|---|---|---|---|---|
| ment | Glucoamylase | Amylase | (mg EP/ | Ethanol (% w/v) | | |
| No. | (AGU/gDS) | (FAU-F/gDS) | gDS) | 20 hr | 47 hr | 70 hr |
| 1 | 0.500 | 0.0475 | 0.000 | 9.49 | 15.21 | 16.50 |
| 2 | 0.500 | 0.0475 | 0.020 | 9.55 | 15.18 | 16.31 |
| 3 | 0.500 | 0.0475 | 0.100 | 9.84 | 15.60 | 16.84 |
| 4 | 0.500 | 0.0475 | 0.500 | 10.39 | 16.62 | 17.89 |

### Example 6

All treatments were evaluated via mini-scale fermentations similar to Example 5 except the corn slurry, which is made by mixing 103.55 g of ground yellow dent corn (with an average panicle size around 0.5 mm) with 24.23 g backset and 122.46 g tap water to total 250 g slurry. The backset is about 16.5 wt. %. Dry solid was measured to be about 37.49 wt. %. The enzyme dose was based on 34.42% DS. The ethanol yield is shown in Table below. As phytase concentration increases, the % (w/v) ethanol yield increases.

| Treat- | | Alpha- | Phytase | | | |
|---|---|---|---|---|---|---|
| ment | Glucoamylase | Amylase | (mg EP/ | Ethanol (% w/v) | | |
| No. | (AGU/gDS) | (FAU-F/gDS) | gDS) | 20 hr | 47 hr | 70 hr |
| 1 | 0.500 | 0.0475 | 0.000 | 9.72 | 15.40 | 16.67 |
| 2 | 0.500 | 0.0475 | 0.020 | 9.69 | 15.49 | 16.75 |
| 3 | 0.500 | 0.0475 | 0.100 | 9.95 | 15.82 | 17.26 |
| 4 | 0.500 | 0.0475 | 0.500 | 10.63 | 16.84 | 18.03 |

### Example 7

All treatments were evaluated via mini-scale fermentations similar to Example 5 except the corn slurry, which is made by mixing 103.55 g of ground yellow dent corn (with an average particle size around 0.5 mm) with 48.20 g backset and 98.37 g tap water to total 250 g slurry. The backset is calculated as 32.89 wt. %. Dry solid was measured to be about 38.75 wt. %. The enzyme dose was based on 34.42% DS. The ethanol yield is shown in Table below. As phytase concentration increases, the % (w/v) ethanol yield increases. By comparing with ethanol yield results in Examples 3, 4 and 5, it is evident that as backset concentration increases, ethanol yield % (w/v) increases. This indicated that some of the residual starch in backset was fermented to ethanol.

| Treat- | | Alpha- | Phytase | | | |
|---|---|---|---|---|---|---|
| ment | Glucoamylase | Amylase | (mg EP/ | Ethanol (% w/v) | | |
| No. | (AGU/gDS) | (FAU-F/gDS) | gDS) | 20 hr | 47 hr | 70 hr |
| 1 | 0.500 | 0.0475 | 0.000 | 9.72 | 15.61 | 17.02 |
| 2 | 0.500 | 0.0475 | 0.020 | 9.51 | 15.37 | 16.85 |
| 3 | 0.500 | 0.0475 | 0.100 | 9.47 | 15.51 | 16.81 |
| 4 | 0.500 | 0.0475 | 0.500 | 10.20 | 16.66 | 18.24 |

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims, in the case of conflict, the present disclosure including definitions will control.

Various references are cited herein, the disclosures of which are incorporated by reference in their entireties. The present invention is further described by the following examples which should not be construed as limiting the scope of the invention.

1. A process for producing a fermentation product from starch-containing material comprising:
(a) saccharifying starch-containing material with an alpha-amylase at a temperature below the initial gelatinization temperature of said starch-containing material; and
(b) fermenting using a fermenting organism,
wherein a phytase is present during step (a) or (b).

2. The process of claim 1, wherein the saccharification and fermentation is carried out sequentially or simultaneously.

3. The process of claim 1, further wherein a protease is present during step (a) or (b) or during simultaneous saccharification and fermentation in combined steps (a) and (b).

4. The process of claim 1, wherein the temperature during simultaneous saccharification and fermentation, or fermentation in step (b) is between 28° C. and 36° C.

5. The process of claim 1, wherein the alpha-amylase is an acid alpha-amylase.

6. The process of claim 1, wherein a carbohydrate-source generating enzyme is present during saccharification in step (a) or simultaneous saccharification and fermentation in combined steps (a) and (b).

7. The process of claim 1, wherein the carbohydrate-source generating enzyme is a glucoamylase, beta-amylase, or maltogenic amylase, or a mixture thereof.

8. The process of claim 1, wherein the fermentation step (b) or one-step fermentation (simultaneous steps (a) and (b)) is carried out for a period of 1 to 250 hours.

8. The process of claim 1, wherein the process is carried out at a pH in the range between 3-7.

9. The process of claim 1, wherein the dry solids content (DS) in step (a) or combined steps (a) and (b) lies in the range from 10-55 wt. %.

10. The process of claim 1, wherein the sugar concentration during fermentation is kept at a level below about 6 wt. % during fermentation.

11. The process of claim 1, wherein a slurry comprising water and starch-containing material is prepared before step (a).

12. The process of claim 11, wherein the aqueous slurry contains from about 1 to about 70 vol. % backset.

13. The process of claim 1, wherein the starch-containing material is prepared by reducing the particle size of starch-containing material to a particle size of around 0.05 to 3.0 mm.

14. The process of claim 1, wherein the starch-containing material is obtained from corn (maize), cobs, wheat, barley, rye, milo, sago, cassava, manioc, tapioca, sorghum, rice or potatoes.

15. The process of claim 1, wherein the starch-containing material is granular starch.

16. The process of claim 1, wherein the starch-containing material is uncooked starch.

17. The process of claim 1, wherein the fermentation product is an alcohol.

18. The process of claim 1, wherein an alpha-glucosidase is present during steps (a) and/or (b).
